(19) United States
(12) Patent Application Publication
(10) Pub. No.: US 2013/0217969 A1
(43) Pub. Date: Aug. 22, 2013

(54) ENDOSCOPE DEVICE

(71) Applicant: FUJIFILM CORPORATION, (US)

(72) Inventors: Nobuyuki TORISAWA, Ashigarakami-gun (JP); Masayuki IWASAKA, Ashigarakami-gun (JP)

(73) Assignee: FUJIFILM CORPORATION, Tokyo (JP)

(21) Appl. No.: 13/767,271

(22) Filed: Feb. 14, 2013

(30) Foreign Application Priority Data
Feb. 22, 2012 (JP) 2012-035915

(51) Int. Cl. A61B 1/12; A61B 1/00

(52) U.S. Cl. CPC A61B 1/126; A61B 1/00135
USPC 600/121; 600/157

(57) ABSTRACT

In a tip face of an insertion part of an endoscope, gas is discharged from a plurality of air supply ports formed around an observation window, and air currents are generated. This removes suspended matter, such as oil particles and fat particles, from an imaging visual field. The interval between adjacent air supply ports of the air supply port is specified so that the gas around air currents is caught in the air currents by the air currents without being directed towards the imaging lens. The surrounding gas by the air currents being discharged is pulled in the imaging direction along with the air currents. Since the surrounding gas induced by the air currents is prevented from being induced in the direction of the imaging lens, the imaging lens can be prevented from being contaminated by an unnecessary object.

### BACKGROUND OF THE INVENTION

1. Field of the Invention

The present invention relates to an endoscope device that is inserted into the inside of the body to image the inside of the body.

2. Description of the Related Art

In a rigid endoscope device, a rigid endoscope is inserted into a living body to view an image inside of the living body. A tip face of the rigid endoscope is provided with an imaging lens, and an object whose image is formed by the imaging lens is imaged by an imaging device built in a tip portion of the rigid endoscope. Since the rigid endoscope is inserted into the inside of the living body, blood, mucus, or the like may adhere to the imaging lens and may obstruct a visual field. For this reason, there is one that cleans the imaging lens (JP 1993-199979A (JP-H5-199979A)).

Additionally, in the treatment of the rigid endoscope using an electro-cautery device, the visual field may be obstructed due to smoke generation or the like. Therefore, one that releases inert gas harmless to a human body jetted forward from the tip face is also inside the rigid endoscope (JP3310349B). FIG. 16 is a front view showing the tip face of an insertion part 120 of that kind of rigid endoscope. The tip face of the rigid endoscope insertion part 120 is provided with an observation window 124. Jet nozzles 121, 122, and 123 that jet gas forward are formed around the observation window 124.

### SUMMARY OF THE INVENTION

In the endoscope device described in JP 1993-199979A (JP-H5-199979A), for example, in a case where a targeted part or the like is burned off for observation using the endoscope, measures against problems of suspended matter, such as oil particles and fat particles or steam, which is generated by the burning, may float between an object to be observed and the imaging lens, whereby the field of view of the imaging device may be obstructed, are not considered.

In the endoscope device described in JP3310349B, as shown in FIG. 16, air currents wi and wo, shown by arrows, that are induced by the jetting of the gas are generated in a case where gas is jetted from the jet nozzles 121, 122, and 123 (gas is jetted perpendicular to the sheet plane). Although some air current wi is pulled by the gas jetted from the jet nozzles 121, 122, and 123 and flows to the front of the rigid endoscope insertion part 120 along with gas, the other air current wo may be pulled into the surface of the observation window 124. Then, suspended matter, such as oily particles, will adhere to the surface of the observation window 124. In this way, in the endoscope described in JP3310349B, if gas is jetted from the jet nozzles 121, 122, and 123 in order to improve the field of view of the imaging device, suspended matter may adhere to the surface of the observation window 124, and the field of view may become poor instead.

The present invention has been made in view of the above-mentioned problems and an object of the present invention is to prevent dirt from adhering to an imaging window in a case where gas is discharged forward in order to improve a field of view.

The endoscope device according to the present invention is an endoscope device including an insertion part having a tip portion located on a tip side, a base end portion located on the a end side, and a longitudinal axis located between the tip portion and the base end portion; an observation window formed in the tip portion of the insertion part; a first air supply port formed at the periphery of the observation window, to discharge gas in an observation direction from the observation window; and a second air supply port formed at a position adjacent to the first air supply port at the periphery of the observation window to discharge gas in the observation direction from the observation window. The shortest distance between an opening edge of the first air supply port and an opening edge of the second air supply port is equal to or less than a total distance of a shortest distance from an edge of a region where the gas caught in by the gas discharged from the first air supply port is present to the opening edge of the first air supply port and an shortest distance from an edge of a region where the gas caught in by the gas discharged from the second air supply port is present to the opening edge of the second air supply port.

According to the present invention, the first air supply port and the second air supply port are formed around the observation window. Thus, even when suspended matter is present between an object to be observed and the imaging window, and the field of view becomes poor; the suspended matter can be removed from the field of view by the gas discharged from the first air supply port and the second air supply port. Particularly, in this invention, the shortest distance between the opening edge of the first air supply port and the opening edge of the second air supply port adjacent to the first air supply port is equal to or less than the total distance of the shortest distance from the edge of a region where the gas caught in by the gas discharged from the first air supply port is present to the opening edge of the first air supply port and the shortest distance from the edge of a region where the gas caught in by the gas discharged from the second air supply port is present to the opening edge of the second air supply port. Thus, the surrounding gas induced by the gas discharged from the first air supply port and the second air supply port is not guided to the observation window, but is pulled in the observation direction through the observation window by the gas discharged from the first air supply port and the second air supply port. For this reason, when gas is discharged from the first air supply port and the second air supply port, the suspended matter carried by the surrounding gas induced by the gas discharged from the first air supply port and the second air supply port will not also flow to the observation window. As a result, the suspended matter can be prevented from adhering to the observation window.

The opening portion shape of the first air supply port and the second air supply port is, for example, a circle, or an ellipse or an oval having a major axis in the circumferential direction of the observation window.

The endoscope device may include a first air supply tube that guides the gas, which is discharged from the first air supply port, to the first air supply port, and a second air supply tube that guides the gas, which is discharged from the second air supply port, to the second air supply port. In this case, the first air supply tube and the second air supply tube may be put together into one air supply tube on the base end side.

The endoscope device may further include an overtube mounted around the insertion part, and a first projection and a second projection that protrude into the inside of the overtube may be formed at positions that are different in the circumferential direction in the tip portion of the overtube. In this case, the first air supply port and the second air supply port are clearance gaps between the first projection and the second projection in the circumferential direction.

Preferably, a height of the first projection and a height of the second projection are the same.

The endoscope device is, for example, a rigid endoscope or a soft endoscope.

The endoscope device may further include an air supply device that supplies the gas, which is discharged from the first air supply port and the second air supply port, to the first air supply port and the second air supply port.

The first air supply port and the second air supply port may be formed in the tip face of the insertion part.

The first air supply port and the second air supply port may be positioned so that regions where the gas caught in by the gas discharged from the first air supply port and the second air supply port is present and come in contact with each other or overlap each other.

### BRIEF DESCRIPTION OF THE DRAWINGS

FIG. 1 is a perspective view of an endoscope insertion part.

FIG. 2A is a side view of the endoscope insertion part, and FIG. 2B is a front view of the endoscope insertion part.

FIG. 3A is an end view along line III-III of FIG. 2B, and FIG. 3B is a front view of the endoscope insertion part.

FIG. 4 is a front view of an air supply port.

FIG. 5 is a front view of the endoscope insertion part.

FIG. 6 shows a situation in which a rigid endoscope is used.

FIG. 7 shows a situation in which air is supplied from the endoscope insertion part.

FIG. 8 is a perspective view of the endoscope insertion part.

FIG. 9 is a front view of the endoscope insertion part.

FIG. 10 is a side view of the endoscope insertion part.

FIG. 11 is a perspective view of an overtube.

FIG. 12 is a front view of the overtube.

FIG. 13 is a perspective view of the endoscope insertion part and the overtube.

FIG. 14 is a perspective view of the endoscope insertion part mounted with the overtube.

FIG. 15 is a front view of the endoscope insertion part mounted with the overtube.

FIG. 16 is a front view of the endoscope insertion part.

### DESCRIPTION OF THE PREFERRED EMBODIMENTS

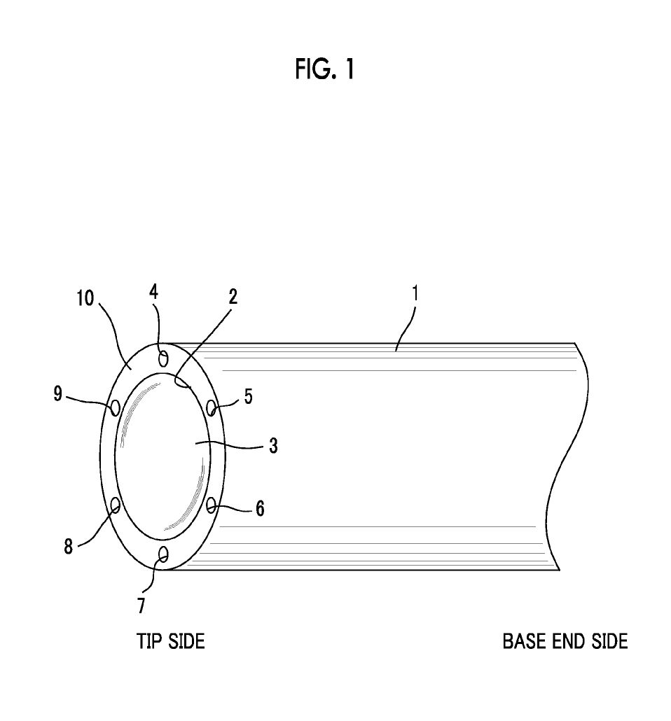

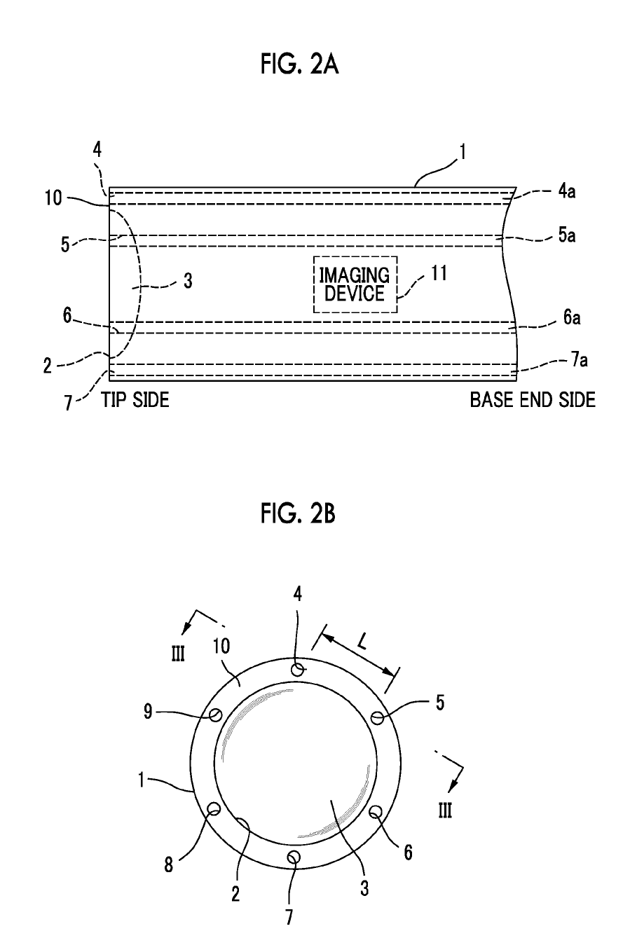

FIGS. 1 to 3B show an embodiment of the present invention and show a tip portion of an endoscope insertion part 1 of a rigid endoscope. FIG. 1 shows a perspective view of the tip portion of the endoscope insertion part 1, FIG. 2A is a side view of the tip portion of the endoscope insertion part 1, and FIG. 2B is a front view of the endoscope insertion part 1.

The endoscope insertion part 1 is inserted into the inside of the body, and has a substantial columnar longitudinal axis. A tip face 10 of the endoscope insertion part 1 is formed with a circular observation window 2 that is slightly smaller than the circular shape of the tip face 10. An imaging lens 3 is fitted into the observation window 2. In the present embodiment, six circular air supply ports 4 to 9 are equally formed in the circumferential direction around the observation window 2. However, the air supply ports are not necessarily equally formed. Additionally, in the present embodiment, six air supply ports 4 to 9 are formed. However, the number of air supply ports may not necessarily be six if the conditions to be described below are satisfied.

With reference to, mainly, FIG. 2A, an air supply tube connected to the six air supply ports 4 to 9 are formed in the longitudinal direction of the endoscope insertion part 1. Although the air supply tubes 4a, 5a, 6a, and 7a connected to the air supply ports 4, 5, 6, and 7 are shown in FIG. 2A, the air supply tubes are also formed in the other air supply ports 8 and 9.

As shown in FIG. 2A, an imaging device 11 for imaging an examination region from the observation window 2 is contained in the tip portion of the endoscope insertion part 1.

Referring to FIG. 2B, the shortest distance L from the edge of the first air supply port 4 to the second air supply port 5 adjacent to the first air supply port 4 among the air supply ports 4 to 9, as will be described below in detail, is made equal to or less than the total distance of the shortest distance from the edge of a region where the gas caught in by discharging gas from the first air supply port is present to the edge (edge nearest to the second air supply port 5) of the first air supply port 4 and the shortest distance from the edge of a region where the gas caught in by discharging gas from the second air supply port is present to the edge (edge nearest to the first air supply port 4) of the second air supply port 5. The other air supply ports 6 to 9 are also adapted to have the positional relationship between the first air supply port 4 and the second air supply port 5.

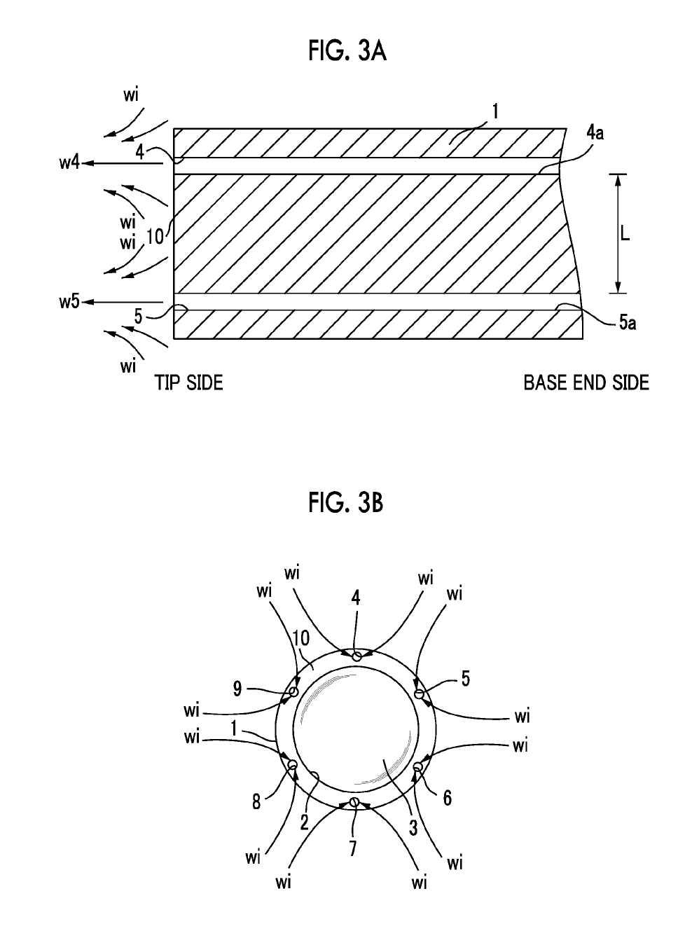

FIG. 3A is an end view along line III-III of FIG. 2B. FIG. 3A is enlarged in comparison to FIG. 2A. FIG. 3B is a front view of the endoscope insertion part 1, which corresponds to FIG. 2B.

Referring to, mainly, FIG. 3A, if gas is supplied from the air supply tubes 4a and 5a, air currents w4 and w5, shown by arrows, are generated toward an observation direction through the observation window 2 from the air supply ports 4 and 5. Then, an air current wi, shown by arrows, is generated by the air currents w4 and w5. The generated air current wi is caught in by the air currents w4 and w5, and moves in the longitudinal direction together with the air currents w4 and w5. As shown in FIG. 3B, a surrounding gas around the air supply port 4 to 9 moves forward in the longitudinal direction while being pulled in the direction of the air supply port 4 to 9, as shown by the air current wi.

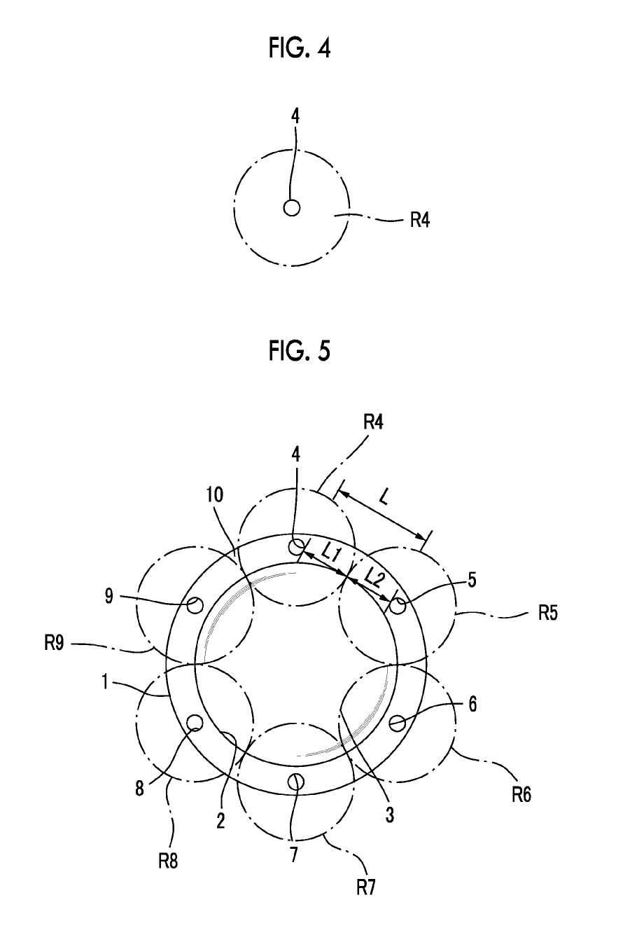

FIG. 4 is a front view of the air supply port 4.

As described above, in a case where gas is discharged from the air supply port 4, the surrounding gas of the air supply port 4 is also caught in by the discharged gas, and is moved to the front of the air supply port 4. In this way, in a case where gas is discharged from the air supply port 4, the range where the gas caught in by the discharged gas is present is shown by a chain line R4. The range R4 is specified according to the amount and velocity of the gas discharged from the air supply port 4. This range R4 (other ranges R5 to R9 to be described below are also the same) can be determined by trial and error while actually discharging gas, or can also be determined by performing a simulation.

FIG. 5 shows the relationship between the air supply ports 4 to 9 formed in the tip face 10 of the endoscope insertion part 1, and the ranges R4 to R9 of the surrounding gas caught in by the gas discharged from the air supply ports 4 to 9.

As described above, if gas is discharged from each of the air supply ports 4 to 9, the surrounding gas that is present within the ranges R4 to R9 centered on the air supply ports 4 to 9 is caught in by the gas discharged from the air supply ports 4 to 9, and is moved forward. The gas that is present outside the ranges R4 to R9 does not enter the ranges surrounded by the ranges R4 to R9. For this reason, even when particles or the like of fats and oils are in the surrounding gas, the particles are caught in by the gas discharged from the air supply ports 4 to 9 by supplying air from the air supply ports 4 to 9. Thus, the particles or the like can be prevented from adhering to the surface of the imaging lens 3.

As described above, in the present embodiment, the shortest distance between the edges of air supply ports adjacent amongst the air supply ports 4 to 9 is made equal to or less than the total distance of the shortest distance from the edge of a range where the gas caught in by the gas discharged from the adjacent air supply ports is present to these air supply ports. For example, the shortest distance L between the opening edge of the air supply port 4 and the opening edge of the air supply port 5 is set to the total distance or less of the shortest distance L1 from the edge of a region (range R4) where the gas caught in by the gas discharged from the air supply port 4 is present to the opening edge of the air supply port 4 and the shortest distance L2 from the edge of a region (range R5) where the gas caught in by the gas discharged from the air supply port 5 is present to the opening edge of the air supply port 5. As for the relationship between the other air supply ports 6 to 9 and the ranges R6 to R9 where the gas caught in by the gas discharged from the air supply ports 6 to 9 is present, similarly, the shortest distance between adjacent air supply ports is made equal to or less than the total distance of the shortest distance from the edge of a range where the gas caught in by the gas discharged from the adjacent air supply ports is present to these adjacent air supply ports. The positions of the air supply ports 4 to 9 may be determined that the above-described ranges R4 to R9 covers the entire surface of the imaging lens 3 (observation window 2).

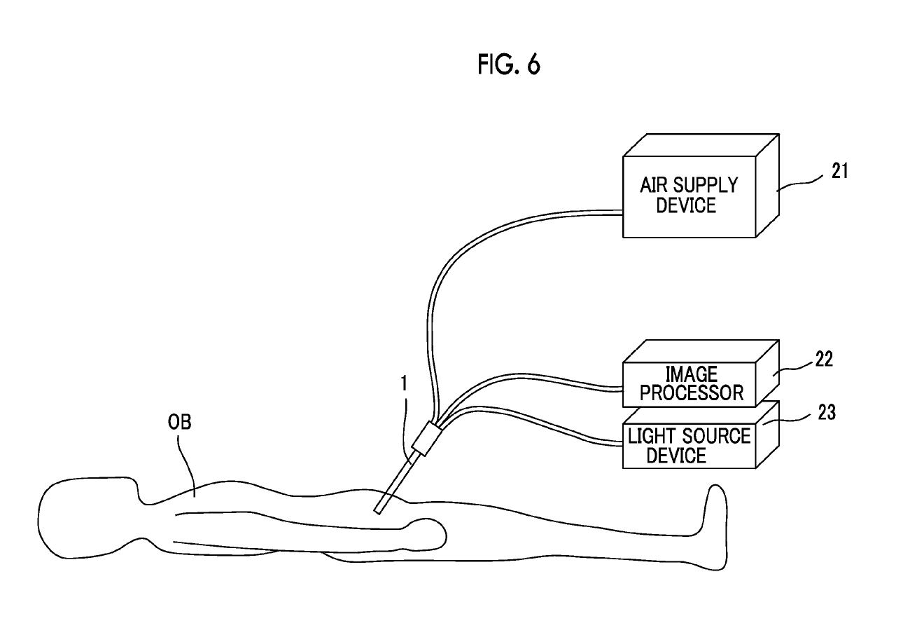

FIG. 6 shows the embodiment of the present invention and shows the outline of the endoscope device.

As described above, a columnar endoscope insertion part (endoscope) 1 inserted into the abdominal cavity of a subject OB is included in the endoscope device. An air supply device 21 that discharges air, an image processor 22, and a light source device 23 that generates a light source that irradiates the inside of the body is connected to the endoscope insertion part 1.

In the endoscope device, the endoscope insertion part 1 is inserted into the abdominal cavity of the subject OB, the inside of the abdominal cavity of the subject OB is imaged, and the captured image is displayed on a display screen. An operator performs an operation while viewing an image displayed on the display screen.

In the present embodiment, the air discharged from the air supply device 21 passes through the inside of the air supply tubes of the endoscope insertion part 1, and is discharged from the air supply ports of the endoscope insertion part 1. Air is supplied from the endoscope insertion part 1 into the abdominal cavity of the subject OB.

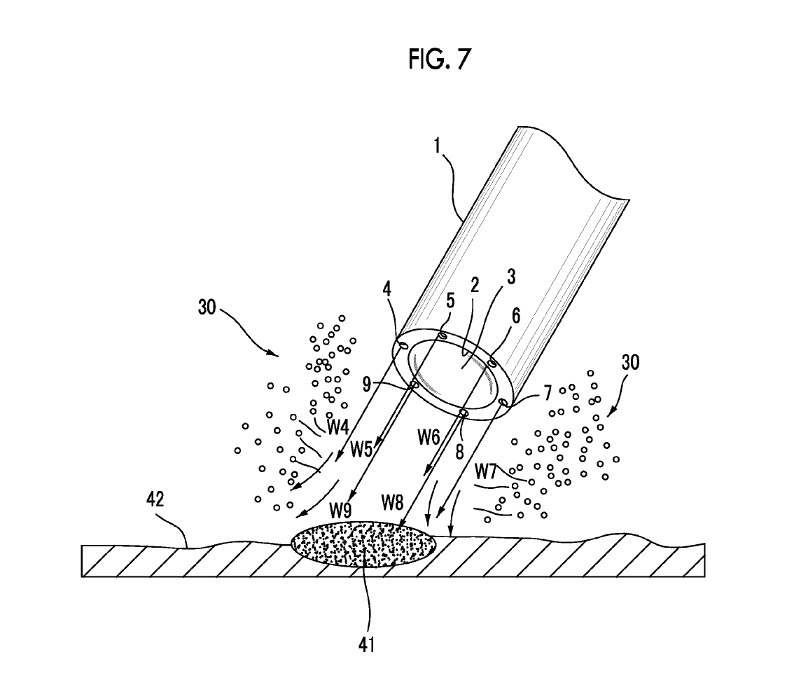

FIG. 7 shows the tip portion of the endoscope insertion part 1 inserted into the abdominal cavity.

As described above, the imaging device is contained in the tip portion of the endoscope insertion part 1, and a part 41 to be observed within the abdominal cavity 42 of the subject OB is imaged (observed) from the observation window 2 of the tip face 10 of the endoscope insertion part 1 (illustration of a window through which light is radiated is omitted).

In the present embodiment, as described above, if gas is discharged in the longitudinal direction from the air supply ports 4 to 9 formed in the tip face 10 of the endoscope insertion part 1, the air currents w4 to w9 are generated, as shown by arrows. In a case where suspended matter 30, such as oil particles and fat particles or steam, is between the observation window 2 and the part 41 to be observed by the air currents w4 to w9, the suspended matter 30 is removed from the imaging range of the imaging device 11. The field of view of the imaging device 11 becomes excellent, and the high-quality image of the part 41 to be observed is obtained. Particularly, in the present embodiment, as described above, the distance between adjacent air supply ports of the air supply ports 4 to 9 is specified so that an air current induced by the air currents w4 to w9 generated by the gas discharged from the air supply port 4 to 9 is not drawn in the imaging lens 3. Therefore, the unnecessary suspended matter 30, such as particles, can be prevented from adhering to the imaging lens 3 of the endoscope insertion part 1.

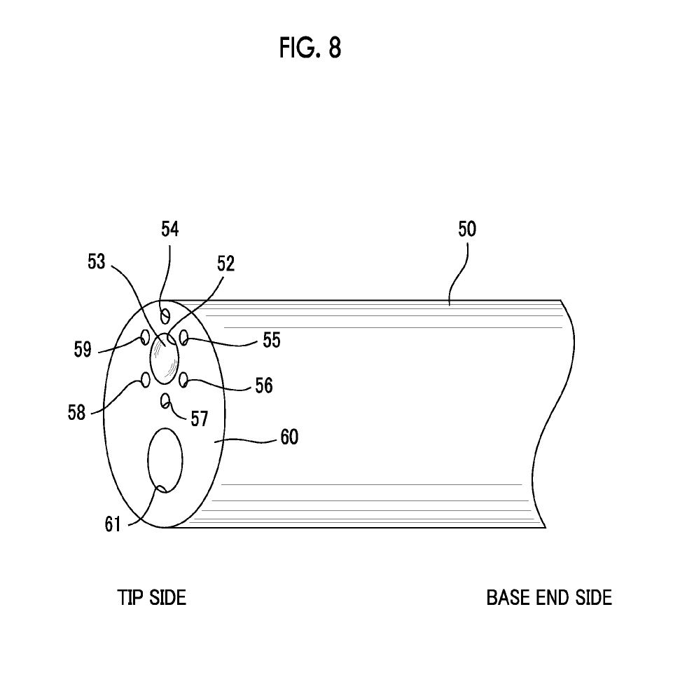

FIG. 8 is a perspective view showing a tip portion of an insertion part 50 of a soft endoscope.

The insertion part 50 of the soft endoscope is also substantially columnar. An imaging window 52 is formed in a tip face 60 of the insertion part 50 of the soft endoscope. An imaging lens 53 is fitted into the imaging window 52. Air supply ports 54 to 59 are formed around the imaging window 52. Additionally, a forceps channel 61 is formed at the tip face 60 of the insertion part 50 of the soft endoscope.

Air is supplied from the air supply ports 54 to 59 formed around the imaging window 52. The distance between adjacent air supply ports is specified, similarly to the air supply ports 4 to 9 formed in the endoscope insertion part 1 of the above-described rigid endoscope. Even in the case of the insertion part 50 of the soft endoscope, similarly to the insertion part 1 of the rigid endoscope, where objects of unnecessary particles are present ahead in the imaging direction, the unnecessary objects in the air can be removed by the air discharged from the air supply ports 54 to 59, and the surrounding gas caught in the air discharged from the air supply ports 54 to 59 is also moved in the imaging direction along with the air discharged from the air supply ports 54 to 59 without being induced to the imaging lens 3.

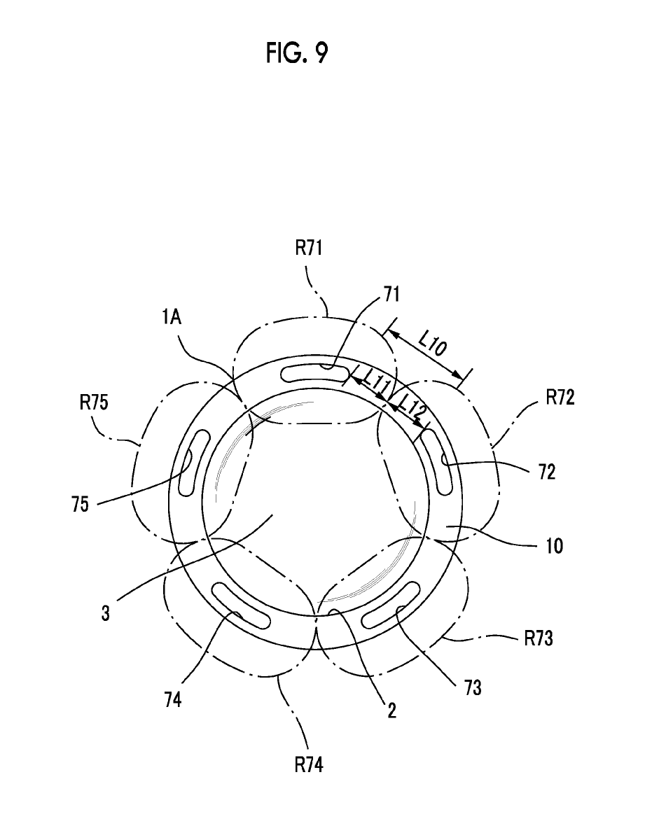

FIG. 9 is a front view of an insertion part 1A of the rigid endoscope.

The tip face 10 of the insertion part 1A of the rigid endoscope is formed with the observation window 2 into which the imaging lens 3 is fitted, as described above. In the present embodiment, there are five oval air supply ports 71 to 75 that are elongated in the circumferential direction around the observation window 2. However, the number of the air supply ports is not necessarily five. Additionally, the ovals 71 to 75 have a major axis in the circumferential direction. Moreover, the shortest distance (L10) between the opening edge of an arbitrary first air supply port (for example, an air supply port 71) that constitute the air supply ports 71 to 75, and the opening edge of the second air supply port (for example, an air supply port 72) adjacent to the first air supply port is equal to or less than the total distance of the shortest distance (L11) from the edge of a region (for example, a range R71) where the gas in which surrounding gas is caught by the gas discharged from the first air supply port is present to the opening edge of the first air supply port, and the shortest distance (L12) from the edge of a region (for example, a range R72) where the gas in which surrounding gas is caught by the gas discharged from the second air supply port is present to the opening edge of the second air supply port. In this way, the distance (distance from the edge of a hole to the edge of another hole) between adjacent ovals of the oval air supply ports 71 to 75 is specified so that ranges R71 to R75 where surrounding gas is caught in when air is discharged from the air supply ports 71 to 75 come in contact with each other (overlap each other) between adjacent ranges.

In this way, the cross-section of the air supply ports may be not only a circle but an oval or an ellipse. Additionally, the cross-section may be rectangles, such as a square or an oblong. In not only the rigid endoscope but the soft endoscope, the cross-section of the air supply ports can have shapes other than the circle.

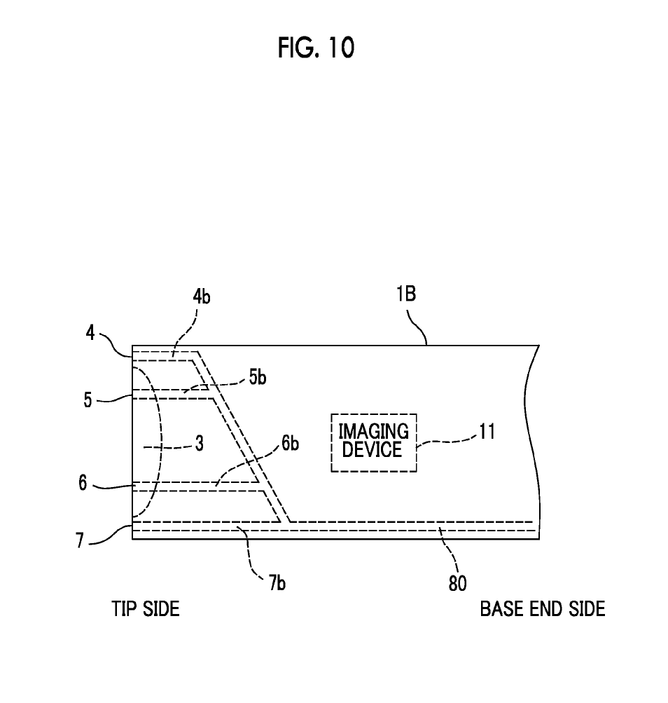

FIG. 10 is a side view of a tip portion of an insertion part 1B of the rigid endoscope.

In the insertion part 1B shown in FIG. 10, air supply tubes (air supply tubes 4b, 5b, 6b, and 7b connected to the air supply ports 4, 5, 6, and 7 are shown in FIG. 10) that supply the air discharged from air supply ports 4 to 9 (only air supply ports 4 to 7 are shown in FIG. 10) are formed on the tip side in one-to-one correspondence to the air supply ports. However, the air supply tubes 4b, 5b, 6b, 7b, and the like are put together into one air supply tube 80 on the base end side.

As air is supplied to one air supply tube 80, air is separately discharged to the air supply tubes 4b, 5b, 6b, 7b, and the like respectively, and is discharged from the air supply ports 4, 5, 6, 7, and the like.

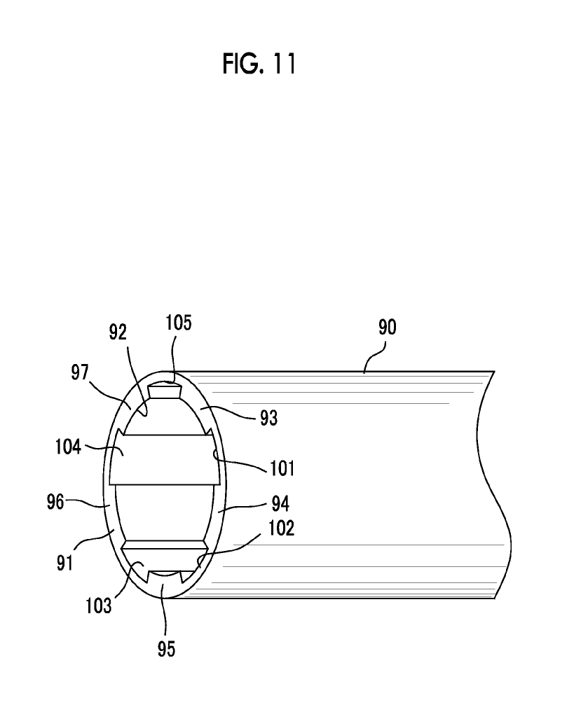

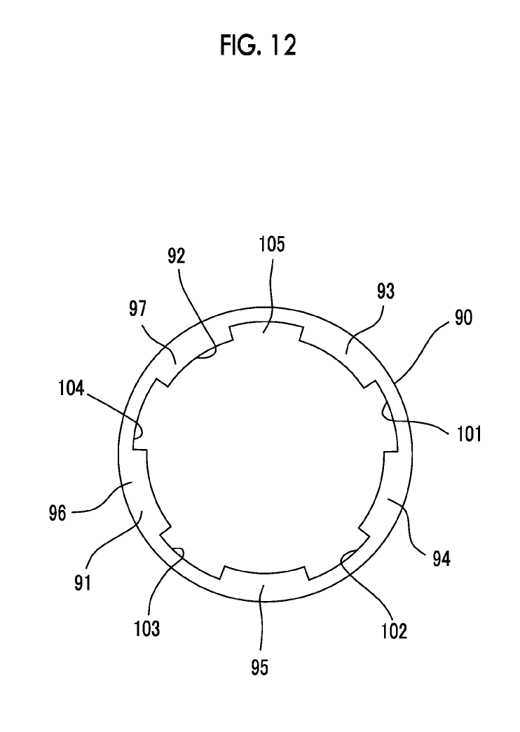

FIG. 11 is a perspective view of an overtube (oversheath) 90, and FIG. 12 is a front view of the overtube.

An opening 92 of a front face of the overtube 90 is formed with five projections 93 to 97 that protrude inwards. Although the height and width of the projections 93 to 97 are the same in the present embodiment, the height and width may not be necessarily the same. Recesses 101 to 105 are formed between the projections 93 to 97, respectively. Such recesses 101 to 105 become the above-described air supply ports.

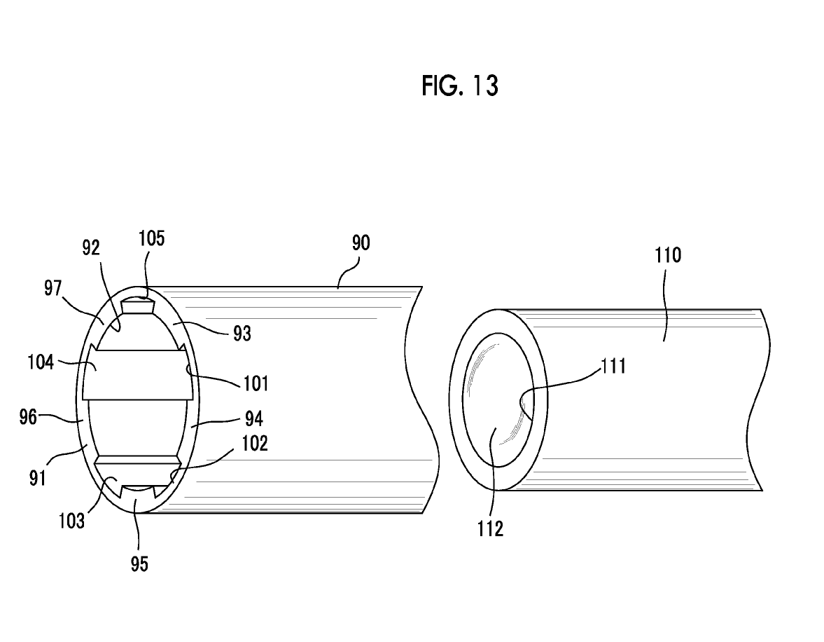

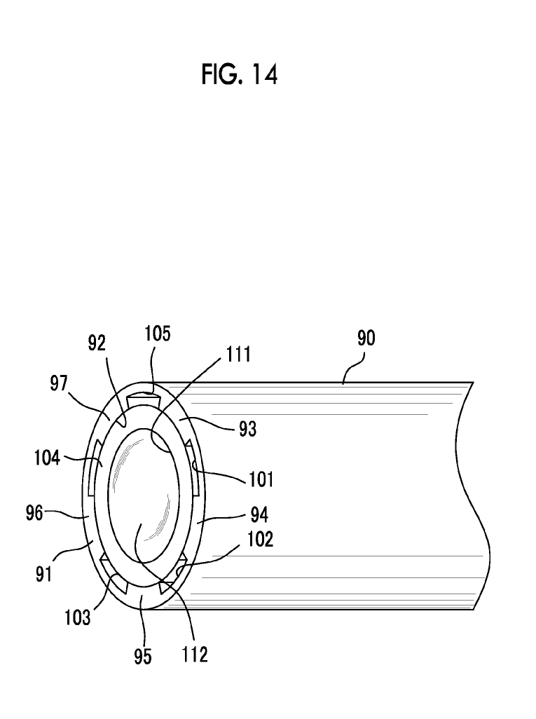

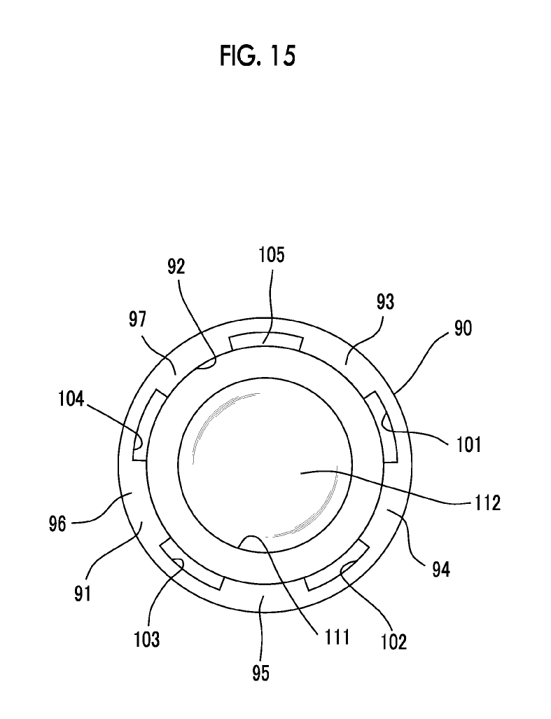

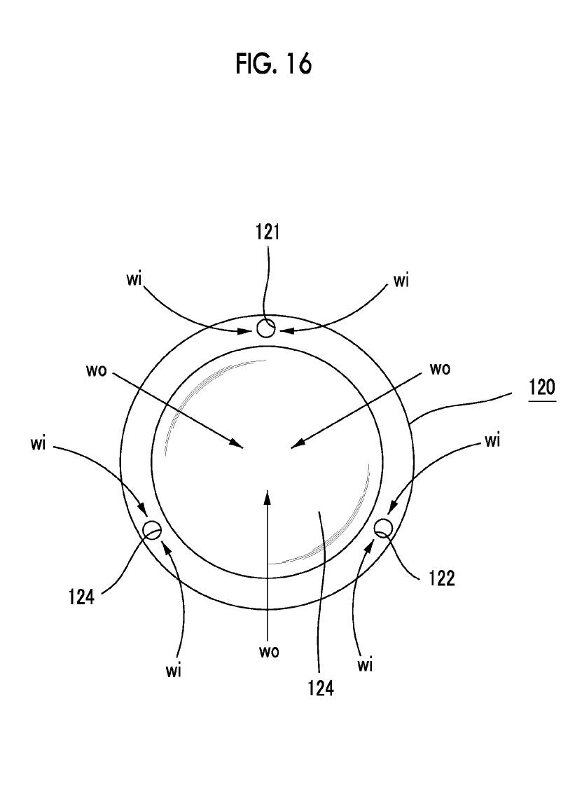

FIG. 13 is a perspective view showing a rigid endoscope 110 and the overtube 90, FIG. 14 is a perspective view showing the rigid endoscope 110 mounted with the overtube 90, and FIG. 15 is a front view showing the mounted rigid endoscope 110 equipped with the overtube.

The tip face of the rigid endoscope 110 is formed with an imaging window 111 into which an imaging lens 112 is fitted.

The internal diameter of the overtube 90 and the external diameter of the rigid endoscope 110 are almost equal, and if the rigid endoscope 110 is mounted with the overtube 90, tops of the projections 93 to 97 of the overtube 90 come in contact with the outer peripheral surface of the rigid endoscope 110. Then, spaces are formed between the outer periphery of the rigid endoscope 110, and the inner peripheral surface of the overtube 90 by the recesses 101 to 105 formed between the projections 93 to 97. The recesses 101 to 105 become the air supply ports by these spaces. If air is supplied from the base end side of the overtube by the air supply device 2 in a state where the rigid endoscope 110 is mounted with the overtube 90, the air is discharged from the recesses 101 to 105 of the tip face.

Even in the present embodiment, as described above, the shortest distance between the opening edge of an arbitrary first recess and the opening edge of a second recess, among the recesses 101 to 105, that are adjacent to each other is specified to the total distance or less of the shortest distance from the edge of a region where the gas caught in by the gas discharged from the first recess is present to the opening edge of the first recess and the shortest distance from the edge of a region where the gas caught in by the gas discharged from the second recess is present to the opening edge of the second recess. It can also be said that the distance between adjacent recesses of the recesses 101 to 105 is specified so that ranges where the surrounding gas generated by discharging air from the recesses 101 to 105 is caught, comes in contact with each other (overlap each other) between adjacent ranges.

As for not only the overtube mounted on the rigid endoscope 110 but the overtube mounted on the soft endoscope, the air supply ports can be similarly formed in the overtube by forming the projections. As for these overtubes, the air supply tubes (spaces between the outer peripheral surface of the endoscope and the inner peripheral surface of the overtube) that guides air to the air supply ports (recesses) may be formed in one-to one correspondence to the air supply port on the tip portion, and may be formed so as to become one toward the base end side. Otherwise, the air supply tubes of a number corresponding to the air supply ports may be formed to the base end side.

What is claimed is:

1. An endoscope device comprising:
an insertion part having a tip portion located on a tip side, a base end portion located on a base end side, and a longitudinal axis located between the tip portion and the base end portion;
an observation window formed in the tip portion of the insertion part;
a first air supply port formed around the observation window, to discharge gas in an observation direction through the observation window; and
a second air supply port formed at a position adjacent to the first air supply port around the observation window to discharge gas in the observation direction through the observation window,
wherein a shortest distance between an opening edge of the first air supply port and an opening edge of the second air supply port is equal to or less than a total distance of an shortest distance from an edge of a region where the gas caught in by the gas discharged from the first air supply port is present, to the opening edge of the first air supply port, and an shortest distance from an edge of a region where the gas caught in by the gas discharged from the second air supply port is present, to the opening edge of the second air supply port.

2. The endoscope device according to claim 1,
wherein the opening shape of the first air supply port and the second air supply port is a circle, or an ellipse or an oval having a major axis in the circumferential direction of the observation window.

3. The endoscope device according to claim 1, further comprising:
a first air supply tube that guides the gas, which is discharged from the first air supply port, to the first air supply port; and
a second air supply tube that guides the gas, which is discharged from the second air supply port, to the second air supply port,
wherein the first air supply tube and the second air supply tube are put together into one air supply tube on the base end side.

4. The endoscope device according to claim 2, further comprising:
a first air supply tube that guides the gas, which is discharged from the first air supply port, to the first air supply port; and
a second air supply tube that guides the gas, which is discharged from the second air supply port, to the second air supply port,
wherein the first air supply tube and the second air supply tube are put together into one air supply tube on the base end side.

5. The endoscope device according to claim 1, further comprising an overtube mounted on the insertion part,
wherein a first projection and a second projection that protrude into the inside of the overtube are formed at positions that are different in the circumferential direction in the tip portion of the overtube, and
wherein the first air supply port and the second air supply port are clearance gaps between the first projection and the second projection in the circumferential direction.

6. The endoscope device according to claim 2, further comprising an overtube mounted on the insertion part,
wherein a first projection and a second projection that protrude into the inside of the overtube are formed at positions that are different in the circumferential direction in the tip portion of the overtube, and
wherein the first air supply port and the second air supply port are clearance gaps between the first projection and the second projection in the circumferential direction.

7. The endoscope device according to claim 3, further comprising an overtube mounted on the insertion part,
wherein a first projection and a second projection that protrude into the inside of the overtube are formed at positions that are different in the circumferential direction in the tip portion of the overtube, and
wherein the first air supply port and the second air supply port are clearance gaps between the first projection and the second projection in the circumferential direction.

8. The endoscope device according to claim 4, further comprising an overtube mounted on the insertion part,
wherein a first projection and a second projection that protrude into the inside of the overtube are formed positions that are different in the circumferential direction in the tip portion of the overtube, and
wherein the first air supply port and the second air supply port are clearance gaps between the first projection and the second projection in the circumferential direction.

9. The endoscope device described according to claim 5,
wherein a height of the first projection and a height of the second projection are the same.

10. The endoscope device described according to claim 6,
wherein a height of the first projection and a height of the second projection are the same.

11. The endoscope device described according to claim 7,
wherein a height of the first projection and a height of the second projection are the same.

12. The endoscope device described according to claim 8,
wherein a height of the first projection and a height of the second projection are the same.

13. The endoscope device according to claim 1,
wherein the endoscope device is a rigid endoscope or a soft endoscope.

14. The endoscope device according to claim 2,
wherein the endoscope device is a rigid endoscope or a soft endoscope.

15. The endoscope device according to claim 1, further comprising an air supply device that supplies the gas, which is discharged from the first air supply port and the second air supply port, to the first air supply port and the second air supply port.

16. The endoscope device according to claim 2, further comprising an air supply device that supplies the gas, which is discharged from the first air supply port and the second air supply port, to the first air supply port and the second air supply port.

17. The endoscope device according to claim 1,
wherein the first air supply port and the second air supply port are formed in the tip face of the insertion part.

18. The endoscope device according to claim 2,
wherein the first air supply port and the second air supply port are formed in the tip face of the insertion part.

19. The endoscope device according to claim 1,
wherein the first air supply port and the second air supply port are positioned so that regions where the gas caught in by the gas discharged from the first air supply port and the second air supply port is present come in contact with each other or overlap each other.

20. The endoscope device according to claim 2,
wherein the first air supply port and the second air supply port are positioned so that regions where the gas caught in by the gas discharged from the first air supply port and the second air supply port is present, come in contact with each other or overlap each other.
